(19) United States
(12) Patent Application Publication
(10) Pub. No.: US 2017/0000400 A1
(43) Pub. Date: Jan. 5, 2017

(54) Biomarkers For The Prediction Of Treatment Outcomes In ADHD

(71) Applicant: BRC OPERATIONS PTY LIMITED, New South Wales (AU)

(72) Inventor: Evian Gordon, Vaucluse, New South Wales (AU)

(21) Appl. No.: 15/113,757

(22) PCT Filed: Jan. 23, 2015
(86) PCT No.: PCT/AU2015/000038

§ 371 Date: Jul. 22, 2016

(30) Foreign Application Priority Data
Jan. 24, 2014 (AU) 2014900220

(51) Int. Cl. A61B 5/16; A61B 5/00

(52) U.S. Cl. CPC A61B 5/168; A61B 5/4839; A61B 5/7246; A61B 5/7275; A61B 5/162; A61B 5/165

(57) ABSTRACT

The present invention relates to the field of biomarkers and in particular to their utilisation in treatment. Embodiments of the invention have been particularly developed as biomarkers enabling optimisation of treatment regimes and as uses of the biomarkers in tests for the prediction of optimised treatments and treatment outcomes in the treatment of attention-deficit/hyperactivity disorder (ADHD) in children and adolescents. The invention will be described hereinafter with reference to this application. However, it will be appreciated that the invention is not limited to this particular field of use.

### TECHNICAL FIELD

The present invention relates to the field of biomarkers and in particular to their utilisation in treatment. Embodiments of the invention have been particularly developed as biomarkers enabling optimisation of treatment regimes and as uses of the biomarkers in tests for the prediction of optimised treatments and treatment outcomes in the treatment of attention-deficit/hyperactivity disorder (ADHD) in children and adolescents. The invention will be described hereinafter with reference to this application. However, it will be appreciated that the invention is not limited to this particular field of use.

### BACKGROUND

Any discussion of the background art throughout the specification should in no way be considered as an admission that such art is widely known or forms part of common general knowledge in the field.

Attention-deficit/hyperactivity disorder (ADHD) adversely affects 3-10% of school-aged children (APA 2000; Polanzyk et al., 2007). In a longitudinal study in the U.S. known as the National Survey of Children's Health (NSCH), parent-reported ADHD diagnoses (ever) increased from 7.8% to 9.5% during 2003 to 2007 (Blumberg et al., 2012). Children with ADHD experience deleterious cognitive problems, including increased risk of learning disability and communication disorders (Strine et al., 2006). The neurocognitive consequences of ADHD may account for the fact that children with ADHD are at increased risk of experiencing major physical injury and hospitalization (Strine et al., 2006). Data from international studies have indicated that attention deficit problems result in increased involvement in a motor vehicle crash, drinking and driving, and traffic violations (Barkley & Cox, 2007). Given the individual and social burden of ADHD and the availability of effective interventions, facilitating the identification of who will and will not respond to a specific treatment could speed to process of effective intervention.

Although a variety of interventions can reduce the key symptoms of ADHD, stimulant medications, including methylphenidate (MPH), are universally endorsed as the mainstay treatment for most children, adolescents and adults (Pliszka 2007; Sharma & Couture, 2013). Stimulant medication such as MPH is by far the most common first-line treatment of ADHD (Pliszka, 2007) and its efficacy has been consistently demonstrated and replicated in multiple large-scale randomized controlled trials (RCTs) (Greenhill et al., 1996; Greenhill, 2002; MTA Cooperative Group, 1999). Between 2011 and 2012, the prescription of MPH in primary care settings in the US increased by 11%. In England, MPH prescriptions for ADHD in primary care increased by 56% between 2007 and 2013 (Care Quality Commission Report, 2013; http://www.cqc.org.uk/public/publications/surveys/community-mental-health-survey-2013).

Although MPH is generally considered effective in the treatment of ADHD it is noteworthy that only 65% of children and adolescents achieve adequate symptom control when receiving MPH treatment (with individual responses varying) and 35% of children fail to achieve adequate symptom control or present with unacceptable side effects (Kooij et al., 2010).

As such the benefits of ADHD treatment could be significantly improved by identifying children and adolescents who will or will not respond to MPH treatment prior to commencing MPH treatment. Hermens et al. (2006) described the value of finding ways to predict which children and adolescents would be stimulant-responsive. However, currently, there is no objective test for predicting treatment or response outcomes based on a patient's pre-treatment profile and identifying children and adolescents who will or will not respond to MPH treatment has received little attention in the field.

To illustrate the consequences, it is noted that in general practice, presently, “non-responders” are identified through direct medication trial, i.e., by “trial and error”, over a period during which the effectiveness of the stimulant medication of choice (generally MPH) is assessed for each individual patient by monitoring symptom response, side effects, etc. The possibility of having to try a several stimulant medications over a period spanning weeks before any treatment effect is observed places a great burden and stress on a patient and their family while seeking relief from the symptoms of ADHD. As such, an urgent need exists to identify biomarkers which can serve as valid and reliable predictors of treatment outcomes in patients suffering from symptoms of ADHD.

### SUMMARY

It is an object of the present invention to overcome or ameliorate at least one of the disadvantages of the prior art, or to provide a useful alternative.

It is an object of the present invention to provide a test for the prediction of treatment outcomes in children and adolescents suffering from ADHD based on the assessment of cognitive functions useful as prognostic biomarkers for ADHD treatment outcomes.

The present inventor has been involved in an international clinical study in which a large group of ADHD outpatients (children and adolescents aged between 6 and 17) has been examined with a view to identifying predictors for optimised ADHD treatment. The study has been designed as a real-world effectiveness trial, primarily to identify which pre-treatment characteristics could serve as much-needed predictors or moderators of treatment response to MPH.

The goal of the international clinical study, a multisite effectiveness trial of MPH for the treatment of ADHD (target sample N=672), was to identify predictors or moderators of ADHD treatment outcomes that are sufficiently predictive to change how practitioners administer MPH to children and adolescents presenting with ADHD. The present inventor has found surprising, robust relationships between certain pre-treatment patient characteristics and desired treatment outcomes in ADHD patients treated with MPH, the most commonly prescribed, first-line stimulant medication for ADHD.

As will be appreciated, the methods of the present invention allow clinicians to apply brief, low-cost, easily conducted tests when deciding whether MPH treatment should be prescribed for a child or adolescent suffering from ADHD.

Accordingly, the present invention relates to a method of identifying a predictor of treatment outcome in attention-deficit/hyperactivity disorder (ADHD) comprising the steps of:
a) determining the degree of ADHD by measuring symptom scores for each member of a group of subjects with ADHD
   (i) before treatment with methylphenidate (MPH), and
   (ii) after a predetermined period of treatment with MPH,
wherein the comparison of the symptom scores measured before and after treatment with MPH provides a measure of treatment outcome for each member; and
b) assessing at least one cognitive parameter in a group of subjects without ADHD and in said group of subjects with ADHD before treatment with MPH to obtain assessment scores for said parameter and to identify at least one parameter for which the assessment scores obtained are markedly different between said two groups;
c) analysing the assessment scores to establish a correlation between
   treatment outcome as indicated by changes in said symptom scores of step a(i), and a(ii), and
   said at least one parameter identified in (b) above,

wherein, when said at least one parameter identified in (b) above is correlated with a treatment outcome across the group of subjects with ADHD, said parameter is identified as a predictor of MPH treatment outcome in ADHD.

In a first aspect, the present invention provides a method of predicting a treatment outcome in a patient with attention-deficit/hyperactivity disorder (ADHD) comprising the steps of:
a) using a computer to assess a cognitive parameter in said patient thereby obtaining an assessment score for said parameter; and
b) comparing said assessment score of step a) with a reference set of assessment scores for said parameter to establish a correlation between said assessment score with a corresponding assessment score of the reference set, wherein said corresponding assessment score is linked to a treatment outcome in ADHD patients having been treated with a selected stimulant medication,
   wherein said correlation of step b) is used to predict a treatment outcome for said patient with ADHD when treated with the selected stimulant medication.

Typically, the reference set of assessment scores comprises assessment scores for cognitive parameters identified as robust predictors of treatment outcome in ADHD patients having been divided into at least two patient sub-groups based on a comparison of
the assessment scores obtained for at least one parameter before treatment, with
assessment scores obtained for said same parameter in the overall population of matched subjects.

Typically, the stimulant medication is methylphenidate (MPH) and said patient is aged between 6 and 17 years.

In some embodiments the cognitive parameter is assessed by applying a computerised test battery including tests selected from Motor Tapping, Choice Reaction Time, Memory recall, Digit Span, Verbal Interference, Switching of Attention, Continuous Performance Test, Go/No-Go, Maze task and Emotion Identification.

Typically, the computerised test battery is the IntegNeuro™ touch-screen cognitive test battery.

In some embodiments, a first cognitive parameter (such as high performance in the Switching of Attention test) is a predictor of negative treatment outcome for ADHD patients in one of the patient sub-groups when the division into said sub-groups is based on the comparison of at least a second cognitive parameter (such as poor performance in the Continuous Performance Test).

In alternative embodiments, a first cognitive parameter (such as poor performance in the Switching of Attention test) is a predictor of positive treatment outcome for ADHD patients in one of the patient sub-groups when the division into said sub-groups is based on the comparison of at least a second cognitive parameter (such as high performance in the Verbal interference test).

In yet further alternative embodiments, a first cognitive parameter (such as high performance in the Switching of Attention test) is a predictor of positive treatment outcome for ADHD patients in one of the patient sub-groups when the division into said sub-groups is based on the comparison of at least a second cognitive parameter (such as high performance in the Digit Span test).

ADHD is diagnosed using the Attention Deficit/Hyperactivity Disorder Rating Scale IV (ADHD-RS IV; Pappas, 2006) and the Mini International Neuropsychiatric Interview for Children and Adolescents (MINI-KID; Sheehan D V, et al. Reliability and validity of the Mini International Neuropsychiatric Interview for Children and Adolescents (MINI-KID). J Clin Psychiatry 2010; 71(3): 313-326) was used to identify other current and lifetime psychiatric co-morbidities. ADHD symptom severity was assessed using the ADHD-RS IV (clinician rated) and the Conners' Parent Rating Scale—Revised: Long Version (CPRS-R:L; Conners C K. Conners' rating scales—Revised user's manual. North Tonawanda, N.Y.: Multi-Health Systems Inc.; 1997). ADHD-RS IV and CPRS-R:L scores are collected at baseline (i.e. before treatment) and after a predetermined period of treatment with MPH, for example after 6 weeks. In some embodiments, the symptom scores are collected at baseline and after 6 weeks. The ADHD-RS IV and CPRS-R:L scores provide a measure of the degree of ADHD in a subject based on the ADHD symptom severity determined. The scores obtained in the above-mentioned symptom reporting scales are referred to herein as “symptom scores”. Symptom response by week 6 was defined a priori as a reduction in symptom scores on the ADHD-RS-IV scale of 25% or greater.

Treatment outcome in children and adolescents with ADHD is routinely determined by a symptom score according to the clinician-rated ADHD-RS IV and the Conners' Parent Rating Scale—Revised: Long Version (CPRS-R: L), wherein a ≧25% decrease of a symptom score determined before treatment with a selected stimulant medication after 6 weeks of treatment with said selected stimulant medication indicates a treatment response.

In a second aspect, the present invention relates to a method of predicting negative MPH treatment outcome in a patient with attention-deficit/hyperactivity disorder (ADHD) comprising the steps of:
a) using a computer to subject said patient to a battery of cognitive tests assessing cognitive parameters, wherein said battery includes at least the Switching of Attention test, the Maze test and the Verbal Interference test to obtain the patient's assessment scores for at least each of the Switching of Attention, the Maze test and the Verbal Interference test; and
b) comparing said assessment scores of step a) with a reference set of assessment scores for cognitive parameters including assessment scores for at least each of the Switching of Attention, the Maze test and the Verbal Interference test;
   wherein,
(i) when it is established that the patient's assessment score for the Switching of Attention test is below a predetermined percentile of the assessment scores for the Switching of Attention test in the reference set, and
   when the patient's assessment score for the Maze test is below a predetermined percentile of the assessment scores for the Maze test in the reference set or the patient's assessment score for the Verbal Interference test is below a predetermined percentile of the assessment scores for the Verbal Interference test in the reference set; or
(ii) when it is established that the patient's assessment score for the Switching of Attention test is above said predetermined percentile in the assessment scores for the Switching of Attention test in the reference set of (i) above, and
   when the patient's assessment score for the Continuous Performance Test is below a predetermined percentile of the assessment scores for the Continuous Performance Test in the reference set,
      a negative MPH treatment outcome for said patient with ADHD is predicted.

Typically, said patient is aged between 6 and 17 years.

Generally, the predetermined percentile of the assessment scores for the Switching of Attention test in item (i) is below or in item (i)) is above the 50^{th} percentile of the assessment scores for the Switching of Attention test in the reference set, such as below or above the 45^{th} percentile, such as below or above the 40^{th} percentile, such as below or above the 35^{th} percentile, such as below or above the 30^{th} percentile, such as below or above the 25^{th} percentile, or such as below or above the 22^{nd} percentile.

Generally, the predetermined percentile of the assessment scores for the Maze test in item (i) is below the 50^{th} percentile of the assessment scores for the Maze test in the reference set, such as below the 45^{th} percentile, such as below the 40^{th} percentile, such as below the 35^{th} percentile, such as below the 30^{th} percentile, such as below the 25^{th} percentile, such as below the 20^{th} percentile, such as below the 15^{th} percentile, or such as below the 14^{th} percentile.

Generally, the predetermined percentile of the assessment scores for the Verbal Interference test in item (i) is above the 30^{th} percentile of the assessment scores for the Verbal Interference test in the reference set, such a above the 34^{th} percentile, such as above the 35^{th} percentile, such as above the 40^{th} percentile, such as above the 45^{th} percentile or such above the 50^{th} percentile.

Generally, the predetermined percentile of the assessment scores for the Continuous Performance Test in item (ii) is below the 50^{th} percentile of the assessment scores for the Continuous Performance Test in the reference set, such as below the 45^{th} percentile, such as below the 40^{th} percentile, such as below the 35^{th} percentile, such as below the 30^{th} percentile, such as below the 25^{th} percentile, such as below the 20^{th} percentile, such as below the 15^{th} percentile, such as below the 10^{th} percentile, such as below the 5^{th} percentile, such as below the 2nd^{th} percentile, such as below the 1^{st} percentile, or such as below the 0.5^{th} percentile.

In a third aspect, the present invention relates to a method of predicting positive MPH treatment outcome in a patient with attention-deficit/hyperactivity disorder (ADHD) comprising the steps of:
a) using a computer to subject said patient to a battery of cognitive tests assessing cognitive parameters, wherein said battery includes at least the Switching of Attention test, the Verbal Interference test and the Digit Span test to obtain the patient's assessment scores for at least each of the Switching of Attention test, the Verbal Interference test and the Digit Span test; and
b) comparing said assessment scores of step a) with a reference set of assessment scores for cognitive parameters including assessment scores for at least each of the Switching of Attention test, the Verbal Interference test and the Digit Span test;
   wherein,
(i) when it is established that the patient's assessment score for the Switching of Attention test is below a predetermined percentile of the assessment scores for the Switching of Attention test in the reference set, and
   when the patient's assessment score for the Verbal Interference test is below a predetermined percentile of the assessment scores for Verbal Interference test in the reference set; or
(ii) when it is established that the patient's assessment score for the Switching of Attention test is above said predetermined percentile in the assessment scores for the Switching of Attention test in the reference set of (i) above, and
   when the patient's assessment score for the Digit Span test is above a predetermined percentile of the assessment scores for the Digit Span test in the reference set,
      a positive MPH treatment outcome for said patient with ADHD is predicted.

Typically, said patient is aged between 6 and 17 years.

Generally, the predetermined percentile of the assessment scores for the Switching of Attention test in item (i) is below or in item (i)) is above the 50^{th} percentile of the assessment scores for the Switching of Attention test in the reference set, such as below or above the 45^{th} percentile, such as below or above the 40^{th} percentile, such as below or above the 35^{th} percentile, such as below or above the 30^{th} percentile, such as below or above the 25^{th} percentile, or such as below or above the 22^{nd} percentile.

Generally, the predetermined percentile of the assessment scores for the Verbal Interference test in item (i) is below the 50^{th} percentile of the assessment scores for the Verbal Interference test in the reference set, such as below the 45^{th} percentile, such as below the 40^{th} percentile, such as below the 35^{th} percentile, or such as below the 34^{th} percentile.

Generally, the predetermined percentile of the assessment scores for the Digit Span test in item (ii) is above the 30^{th} percentile of the assessment scores for the Digit Span test in the reference set, such a above the 35^{th} percentile, such as above the 40^{th} percentile, such as above the 45^{th} percentile, or such as above the 46^{th} percentile.

In a fourth aspect the present invention relates to a method of treating attention-deficit/hyperactivity disorder (ADHD) in a patient, said method comprising the steps of:
a) using a computer to subject said patient to a battery of cognitive tests assessing cognitive parameters, wherein said battery includes at least the Switching of Attention test, the Verbal Interference test and the Digit Span test to obtain the patient's assessment scores for at least each of the Switching of Attention test, the Verbal Interference test and the Digit Span test;
b) comparing said assessment scores of step a) with a reference set of assessment scores for cognitive parameters including assessment scores for at least each of the Switching of Attention test, the Verbal Interference test and the Digit Span test; and
c) administering methylphenidate (MPH) when:
   (i) it is established that the patient's assessment score for the Switching of Attention test is below a predetermined percentile of the assessment scores for the Switching of Attention test in the reference set, and
      when the patient's assessment score for the Verbal Interference test is below a predetermined percentile of the assessment scores for Verbal Interference test in the reference set; or
   (ii) it is established that the patient's assessment score for the Switching of Attention test is above said predetermined percentile in the assessment scores for the Switching of Attention test in the reference set of (i) above, and
      when the patient's assessment score for the Digit Span test is above a predetermined percentile of the assessment scores for the Digit Span test in the reference set.

Typically, said patient is aged between 6 and 17 years.

Generally, the predetermined percentile of the assessment scores for the Switching of Attention test in item (i) is below or in item (i) is above the 50^{th} percentile of the assessment scores for the Switching of Attention test in the reference set, such as below or above the 45^{th} percentile, such as below or above the 40^{th} percentile, such as below or above the 35^{th} percentile, such as below or above the 30^{th} percentile, such as below or above the 25^{th} percentile, or such as below or above the 22^{nd} percentile.

Generally, the predetermined percentile of the assessment scores for the Verbal Interference test in item (i) is below the 50^{th} percentile of the assessment scores for the Verbal Interference test in the reference set, such as below the 45^{th} percentile, such as below the 40^{th} percentile, such as below the 35^{th} percentile, or such as below the 34^{th} percentile.

Generally, the predetermined percentile of the assessment scores for the Digit Span test in item (ii) is above the 30^{th} percentile of the assessment scores for the Digit Span test in the reference set, such a above the 35^{th} percentile, such as above the 40^{th} percentile, such as above the 45^{th} percentile, or such as above the 46^{th} percentile.

In some embodiments the cognitive parameter is assessed by applying a computerised test battery including tests selected from Motor Tapping, Choice Reaction Time, Memory recall, Digit Span, Verbal Interference, Switching of Attention, Continuous Performance Test, Go/No-Go, Maze task and Emotion Identification.

Typically, the computerised test battery is the IntegNeuro™ touch-screen cognitive test battery.

In the context of the present application the term “attention deficit hyperactivity disorder (ADHD)” includes but is not limited to psychiatric disorders of the neurodevelopmental type in which there are significant problems relating to attention, hyperactivity or impulsivity if diagnosed in accordance with the Diagnostic and Statistical Manual of Mental Disorders IV (DSM-IV published by the American Psychiatric Association (1994). Diagnostic and statistical manual of mental disorders (4^{th} ed). Washington D.C.). As indicated above, ADHD symptom severity of can be rated using any appropriate rating scale including the ADHD-RS IV (clinician rated) and the Conners' Parent Rating Scale—Revised: Long Version (CPRS-R: L) scales.

In the context of the present application the term “biomarker” includes but is not limited to objectively measurable and assessable indicators of a biological process or biological state. Preferably, the biomarkers of the present invention indicate changes in symptom severity of ADHD experienced by a patient in response to treatment. In accordance with the present invention, measurable and assessable cognitive parameters can be biomarkers indicating changes in symptom severity of ADHD.

In the context of the present application the term “predictor of treatment outcome” includes but is not limited to biomarkers as defined above, which have a predictive quality with respect to the treatment outcome in ADHD patients when treated with a stimulant medication. “Biomarkers which have predictive quality with respect to the treatment outcome in ADHD patients” here includes but is not limited to biomarkers which have been shown to be correlated with a change in treatment outcome in ADHD patients.

In the context of the present application the terms “matched” or “matching” refer to statistical matching, which includes, but is not limited to, the control/reference group(s)/set(s) having the same (or closely similar) characteristics/values for the matching variables. As the skilled reader will appreciate, matching variables include, but are not limited to, sex, age to within five years, ethnic group, etc.

In the context of the present application the term “treatment outcome” refers to certain threshold symptom scores measured by any appropriate rating scale, including the clinician rated ADHD-RS IV scale and the Conners' Parent Rating Scale—Revised: Long Version (CPRS-R: L), after treatment when compared to the symptom scores obtained before treatment.

In the context of the present application the term “statistically significant correlation” includes but is not limited to statistical correlations having p-values in a range of ≦0.05 (i.e. p-values of, ≦0.01, ≦0.005, ≦0.001, ≦0.0005, or ≦0.0001), or accuracy/sensitivity/specificity in a range of 0.50 or greater (i.e. 0.55, 0.60, 0.65, 0.70, 0.75, 0.80, 0.85, 0.90, 0.95, 0.99 or greater), depending on the specific analysis and the most relevant values for evaluating the outcome of that analysis.

In the context of the present application the term “symptom score” includes but is not limited to any objective measure of symptom severity in ADHD patients. Preferably, symptom scores are determined in accordance with the Diagnostic and Statistical Manual of Mental Disorders IV (DSM-IV published by the American Psychiatric Association (1994). Diagnostic and statistical manual of mental disorders (4^{th} ed). Washington D.C.) and measured by the clinician rated ADHD-RS IV scale and the Conners' Parent Rating Scale—Revised: Long Version (CPRS-R: L).

In the context of the present application the term “stimulant medication” includes but is not limited to medications which enhanced alertness, awareness, wakefulness, endurance, productivity, and motivation, increased arousal, locomotion, heart rate, and blood pressure, and the perception of a diminished requirement for food and sleep. Preferably, the stimulant medications regulate impulsive behaviour and improve attention span and focus by increasing the levels of, for example, dopamine and norepinephrine. The stimulant medication described here includes but is not limited to methylphenidate (MPH).

In the context of the present application the term “cognitive parameter” includes but is not limited to parameters of general cognition and of emotional cognition. Typically such parameters are general cognitive and emotional cognitive processing skills which can be assessed by tests well-known in the art. Preferably several parameters are assessed simultaneously. A suitable validated computer-based, touch-screen cognitive test battery is the “IntegNeuro™” (Brain Resource Ltd.) cognitive test battery described below.

In the context of the present application the term “a reference set of assessment scores” includes but is not limited to a set of assessment scores for the relevant parameters previously found to be correlated with a symptom score of ADHD thereby providing a reference set of assessment scores being statistically significant predictors of treatment outcome in ADHD. The methods according to the present invention provide for the establishment of such a reference set. Typically, the reference set of assessment scores is the collection of assessment scores obtained from a matched population. The reference set, which can also be an index, is useful in clinical practice to determine de novo ADHD treatment regimes with greater confidence of a beneficial treatment outcome for each individual patient as well as to determine optimized treatment regimes for patients with ADHD already receiving stimulant medication.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Embodiments of the invention will now be described, by way of example only, with reference to the accompanying drawings in which:

FIG. 1 is a consort diagram illustrating the overall sample and providing details as to why certain study participants did not complete the follow-up assessment.

FIG. 2 is a classification diagram illustrating the treatment outcome/response in ADHD patients based on assessment scores for selected cognitive parameters assessed using the validated computer-based, touch-screen cognitive test battery cognitive Test Battery.

### DETAILED DESCRIPTION

Preferred embodiments of the invention will now be described.

In one preferred embodiment the present invention relates to a method of identifying a predictor of treatment outcome in attention-deficit/hyperactivity disorder (ADHD).

ADHD is diagnosed using the ADHD-RS IV and the Mini International Neuropsychiatric Interview for Children and Adolescents (MINI-KID; Sheehan D V, et al. Reliability and validity of the Mini International Neuropsychiatric Interview for Children and Adolescents (MINI-KID). J Clin Psychiatry 2010; 71(3): 313-326) was used to identify other current and lifetime psychiatric co-morbidities. ADHD symptom severity was assessed using the ADHD-RS IV (clinician rated) and the Conners' Parent Rating Scale—Revised: Long Version (CPRS-R: L). ADHD-RS IV and CPRS-R: L scores are collected at baseline (i.e. before treatment) and after a predetermined period of treatment with MPH, for example after 6 weeks. In some embodiments, the symptom scores are collected at baseline and after 6 weeks. The ADHD-RS IV and CPRS-R: L scores provide a measure of the degree of ADHD in a subject based on the ADHD symptom severity determined. The scores obtained in the above-mentioned symptom reporting scales are referred to herein as “symptom scores”. Symptom response by week 6 was defined a priori as a reduction in symptom scores on the ADHD-RS-IV scale of 25% or greater.

The comparison of the symptom scores measured before and after treatment with MPH provides a measure of treatment outcome for each patient. The skilled reader will, of course, understand that such symptom scores can be measured by any applicable diagnostic method for determining the degree of ADHD known in the art and that the statistical comparison of the scores can also be performed by methods commonly known in the art.

Once the baseline symptom scores have been collected, and before any treatment with MPH has commenced, further baseline parameters are established for each of the subjects with ADHD as well as for subjects of a matched control group who do not suffer from ADHD. The parameters assessed in one embodiment are, for example, the subjects' general cognitive and emotional cognitive performance. Again, the skilled addressee will appreciate that the above-described parameters are listed as examples only and that the present invention can be performed by assessing other parameters relevant to ADHD.

### Method

### Study Participants

### Enrolment and Screening.

336 children and adolescents aged between 6 and 17 participated in the current study. Enrolment and screening criteria are summarized in Table 1, broad inclusion and minimal exclusion criteria were used to recruit a representative sample of the general ADHD population who typically receive MPH in routine practice.

TABLE 1
Inclusion and Exclusion Criteria

Inclusion Criteria
Meet DSM-IV criteria for ADHD (primary diagnosis), as determined by a
psychiatrist, physician or clinical psychologist, in conjunction with the
clinical workup undertaken at study entry using The Mini International
Neuropsychiatric Interview for Children and Adolescents (MINI Kid)
Score 6 or more items >1 on the Inattentive or Hyperactive/impulsive
subscales of the ADHD Rating Scale IV (ADHD-RS-IV)
No stimulant medication in the 7 days prior to baseline testing
6-17 years of age
Fluent and literate in English (and/or Dutch in The Netherlands).
Exclusion Criteria
Known contra-indication or intolerance to the use of methylphenidate as
defined in the product package insert (including previous treatment
failure at the highest recommended dose)
Pregnancy or females of child bearing potential not using contraception
Known medical condition, disease or neurological disorder which might,
in the opinion of investigator/s, interfere with assessments in the study or
put ADHD subjects at increased risk when exposed to optimal doses of
the drug treatment.
History of physical brain injury or blow to the head that resulted in loss of
consciousness for 5 minutes within the last two years, or for at least 15
minutes longer than 2 years ago
Known past or present substance dependence, including alcohol as
determined by the MINI Kid
Participation in an investigational study within four months of the
baseline visit in which subjects have received an experimental
drug/device that could affect the primary end points of this study.
Use of any psychological or counselling therapy, or CNS medication that
cannot be washed out prior to baseline visit, or use of any psychological
or counselling therapy between baseline and follow-up week 6 visit.
Participants who, in the opinion of the investigator, have a severe
impediment to vision, hearing and/or hand movement, which is likely to
interfere with their ability to complete the testing batteries.
Participants who, in the opinion of the investigator, are unable and/or
unlikely to comprehend and follow the study procedures and instructions.
Presence of any other co-morbid primary DSM IV disorder.

The study was conducted in accordance with the principles of the “Declaration of Helsinki 2008” (World Medical Association (2008). “World Medical Association Declaration of Helsinki ethical principles for medical research involving human subjects.” 59^{th} WMA General Assembly, Seoul, October 2008. Retrieved 22 Jul. 2013, from http://www.wma.net/en/30publications/10policies/b3/17c.pdf) or the International Conference on Harmonization (ICH) guidelines (Retrieved 22 Jul. 2013 from http://www.ich.org/fileadmin/Public_Web_Site/News_room/C_Publications/ICH_20_anniversary_Value_Benefits_of_ICHfor_Regulators.pdf) and/or in accordance with the laws and regulations of the country in which the research is conducted, including the principles of “Good Clinical Practice” as outlined in the US Code of Federal Regulations. Institutional Review Board (IRB)/Independent Ethics Committee (IEC) approval is obtained prior to participant enrolment at each site.

### Study Sample

Of the starting sample of 336 subjects (mean age=11.9; 72.9% male), 284 returned for a follow-up session at week 6 (mean age=11.98; 73.2% male). Of these, 62% (n=176) showed a response to treatment at week 6.

### Procedure

### Treatment Delivery and Compliance Monitoring

ADHD participants were either treatment naïve or washed out before baseline. ADHD participants were prescribed open-label MPH by their treating paediatrician. The open-label treatment design was intended to maximize participant safety, and is consistent with the study's naturalistic methodology.

Participants continued treatment with MPH until week 6 (for a minimum duration of 4 weeks), while refraining from any other ADHD treatments, including other stimulants, non-stimulant ADHD drugs and non-pharmacological ADHD therapies such as counselling or behaviour therapies.

### Measures

Demographic variables were assessed at screening, and clinical and cognitive measures were evaluated at Baseline (Week 0) and Week 6 (post-treatment).

### ADHD Diagnosis, Co-Morbidities, and Symptom Severity

As indicated above, ADHD diagnosis was assessed using the ADHD-RS IV and the MINI Kid was used to identify other current and lifetime psychiatric co-morbidities. ADHD symptom severity was assessed using the ADHD-RS IV and the CPRS-R: L. Both scales were administered at baseline and at week 6. Inter-rater reliability training for administration of the ADHD-RS-IV was provided by the Global Trial Manager to all principal investigators and research staff performing assessments. Participating clinicians received training and were assessed using videorecordings of a simulated consultation between a clinician and a parent describing a child's symptoms. Symptom response by week 6 was defined a priori as a reduction in symptoms on the ADHD-RS-IV of 25% or greater.

Cognitive Measures

Ten cognitive tasks, summarized in Table 2, were administered to participants at baseline and at the study endpoint (Week 6) using the validated computer-based, touch-screen cognitive test battery, “IntegNeuro™” (Brain Resource Ltd.; see Williams et al., Pediatr Neurol 2010; 42(2): 118-126, for a detailed description). Standardized, pre-recorded task instructions will be concurrently presented visually on the screen and audibly through headphones.

TABLE 2
45-minute Battery of Computerized Tests of Cognitive and Emotional Processes

| | Cognitive | |
|---|---|---|
| | capacity | |
| Task name | assessed | Task Description |
| Motor tapping | Motor | Participants repeatedly tap a circle on the screen with the |
| | coordination | index finger of their dominant hand as fast as possible for |
| | | 60 seconds, and then repeat with their non-dominant hand. |
| | | Total task duration is approximately 2 minutes. |
| Choice | Processing | Participants touch one of four circles that lights up, as fast |
| Reaction | speed | as possible. There are 20 pseudo-random trials with a |
| Time | | random delay of 2 to 4 seconds following the previous |
| | | response. Task duration is approximately 2 minutes |
| Memory | Recall memory | Words are presented aurally through headphones and |
| Recall | | participants recall the words by speaking into a |
| | | microphone. Immediate recall is assessed over 4 |
| | | consecutive repeated trials of a list of 12 words being |
| | | presented then recalled. Interference is assessed through a |
| | | fifth trial with new words, and a sixth trial of the original 12 |
| | | word list immediately following. Delayed recall of the |
| | | original list is assessed after 20 minutes. Total task duration |
| | | is approximately 11 minutes. |
| Digit Span | Working memory | Participants recall in sequential order a series of digits that |
| | | are presented one at time on the screen, using a 9-digit |
| | | touchpad that appears after the list is presented. The |
| | | sequence length begins at 3 digits and increases |
| | | progressively throughout the task to a maximum of 9 digits. |
| | | Maximum task duration is approximately 6 minutes. |
| Verbal | Controlled | Coloured words with incongruent colour-word combinations |
| interference | attention | are presented on the screen. Participants are required to |
| | | firstly identify the name (part 1) and then the colour (part 2) |
| | | of each word. Assesses aspects of inhibition and |
| | | interference comparable to those indexed by the Stroop |
| | | test. Task duration is approximately 2 minutes. |
| Switching of | Flexibility | 13 digits (1-13) and 12 letters (A-L) are presented spatially |
| Attention | | across the screen. Participants select responses in |
| | | ascending sequential order, alternating between digits and |
| | | letters (e.g. 1-A-2-B). Task duration is approximately 3 |
| | | minutes. |
| Continuous | Sustained | An n-back continuous performance task in which one of 4 |
| Performance | attention | letters (B, C, D, G) is presented one at time in pseudo- |
| Test | | random sequence, and participants respond when the |
| | | same letter appears twice in row (tapping a circle on the |
| | | screen). Task duration is approximately 6 minutes. |
| Go/No-Go | Inhibition | Participants respond as quickly as possible to the word |
| | | “press” when it is presented in GREEN, and inhibit this |
| | | response when presented in RED. Participants tap a circle |
| | | on the screen to respond. Task duration is approximately 4 |
| | | minutes. |
| Maze | Executive | Participants are required to identify by trial and error a |
| | function | hidden path within an 8 × 8 grid of circles, with a total of 24 |
| | | correct moves required to move from start to finish. Task |
| | | ends with two consecutive error-free path completions, or |
| | | times out after 10 minutes. |
| Explicit | Emotion | Participants identify the emotional expression of faces |
| Emotion | identification | presented on the screen, selecting 1 of 6 word labels |
| Identification | | presented below the face (Happy, Fear, Sad, Anger, |
| | | Disgust, Neutral), with 48 faces in total (8 of each emotion). |
| | | Task duration is approximately 4 minutes. |

Motor Tapping assessed basic motor function, fine movement speed, and manual dexterity (Gill, 1986).
Choice Reaction Time assessed basic sensory-motor functions, visual-motor coordination, information processing speed, speed-accuracy trade-off and mapping of stimulus identification to the appropriate response (Adam et al., 1999).
Memory recall tested immediate and delayed recall scores (i.e., the number of words correctly recalled across the learning trials and the delayed trial), assessing verbal learning, memory recall, and verbal self-monitoring (Crossen, 1994).
Digit Span evaluated immediate recall and assessed the ability to hold, retain and manipulate new verbal information online (Groth-Marnat and Baker, 2003).
Verbal Interference (Word, Colour) measured the ability to inhibit inappropriate, well-learned, impulsive, automatic responses (Sacks, 1991).
Switching of Attention (Digits+Letters) evaluated the ability to sustain and control the direction of attention and switch attention from one over-learned task to another (O'Donnell et al., 1994).
Continuous Performance Test (n-back) assessed the ability to maintain sustained attention and inhibit impulsive responding over an extended period. Additionally, the task assessed target detection, and the ability to update information held in short term memory (Borgaro, 2003).
Go/No-Go assessed executive functioning and cognitive inhibition, or the ability to suppress well-learned, automatic responses (Logan et al., 1984).
Maze task measured how quickly a participant learned the route through the maze and their ability to remember that route. This task involves: executive functioning and planning; the ability to choose, try, reject and adapt alternative courses of thought and action; visuospatial learning and memory (Bowden, 1989).
Emotion Identification measured emotional recognition and discrimination between emotions (Mathersul et al. 2009; Williams et al. 2009).

A total of 48 individual measures derived from these ten tasks were included in the current analysis, using standardized z-scores for each task measure, derived from the normative population scores that form part of the test.

### Responder Criterion

The primary outcome variable was non-response, based on change scores from baseline to week 6 on the ADHD-RS IV. Participants who had less than 25% reduction in baseline ADHD-RS total score were deemed to be non-responders.

### Data Analysis

ROC analyses, based on signal detection methods (Kraemer et al., 1999) were used to identify which variables, and at what level or score, optimally discriminate non-responders and responders. In this recursive partitioning procedure, cut-points on tests are identified that discriminate non-responders from responders at p<0.01. A kappa statistic is calculated for each cut-point, and the largest kappa coefficients correspond to cut-points with maximum sensitivity and specificity (Kraemer et al., 1999; QROC freeware available at mirecc.stanford.edu). This recursive partitioning method uses the patients' cognitive test results to identify subgroups with significantly increased or decreased probability of MPH response. This process is repeated in a classification tree approach, until no further significant partitions can be achieved or until subgroup size reduces to 10 subjects, up to a maximum of 3 times for each progressive tree branch, resulting in a maximum of 8 potential final subgroups at the bottom of the classification tree. For subgroups at the end of each branch of the classification tree, in which the proportion of responders differed from the response rate observed in the overall sample (62%) by at least 10 percentage points either direction (i.e., 72% or greater=elevated response likelihood; 52% or lower=decreased response likelihood), these subgroups are considered to represent “High” and “Low” response groups, respectively. To evaluate the predictive utility of the cognitive test battery, each of the subgroups representing High and Low response were combined and further descriptive statistics were provided. Sensitivity, Specificity, Positive Predicted Value, and Negative Predicted Value were calculated to evaluate the predictive utility of the cognitive test battery. Participants were included if they had missing data on any of the cognitive tests but were excluded if they did not have observed outcome data on the ADHD-RS-IV.

### Results

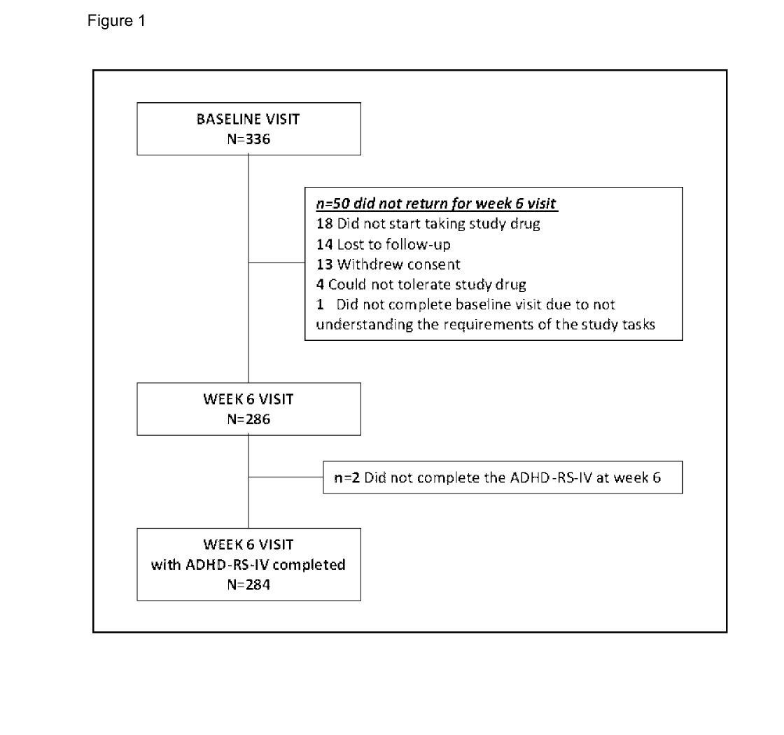

Descriptive statistics of the study sample (n=336) shown in Table 3. Of the total sample who completed the week 6 follow-up assessment (n=284), 62% (n=176) met the criterion for response to methylphenidate (i.e., ≧25% reduction in baseline ADHD-RS IV scores by week 6 endpoint assessment). Details for those participants who did not complete the follow-up assessment are provided in FIG. 1.

Among all participants, 57% reported prior (lifetime) use of stimulant medication to treat ADHD (although washed out at baseline if they had been recently using stimulant mediation). Previous use of stimulant medication was not related to response status at Week 6 (χ²=1.8, df=1, p>0.05).

TABLE 3
Baseline Characteristics of the Study Sample^{a} (Total n = 284).

| | | |
|---|---|---|
| Baseline Characteristic | Mean | SD |

| | | |
|---|---|---|
| Age | 11.9 | 3.2 |
| ADHD-RS-IV Scale Total score (sum of items 1 to | 36.9 | 10.2 |
| 18) | | |
| Conners Parent CGI Total Subscale (T score) | 73.2 | 12.6 |
| Gender (Number of Males) | 208 | 73% |
| ADHD-RS-IV Combined Subtype | 161 | 567% |
| ADHD-RS-IV Inattentive Subtype | 98 | 34% |
| ADHD-RS-IV Hyperactive Subtype | 7 | 2% |
| Below criteria on ADHD-RS-IV | 18 | 6% |

^{a}Data above reflect the sample of subjects, who completed a week 6 follow-up visit.

### Classification of Responders and Non-Responders Via ROC

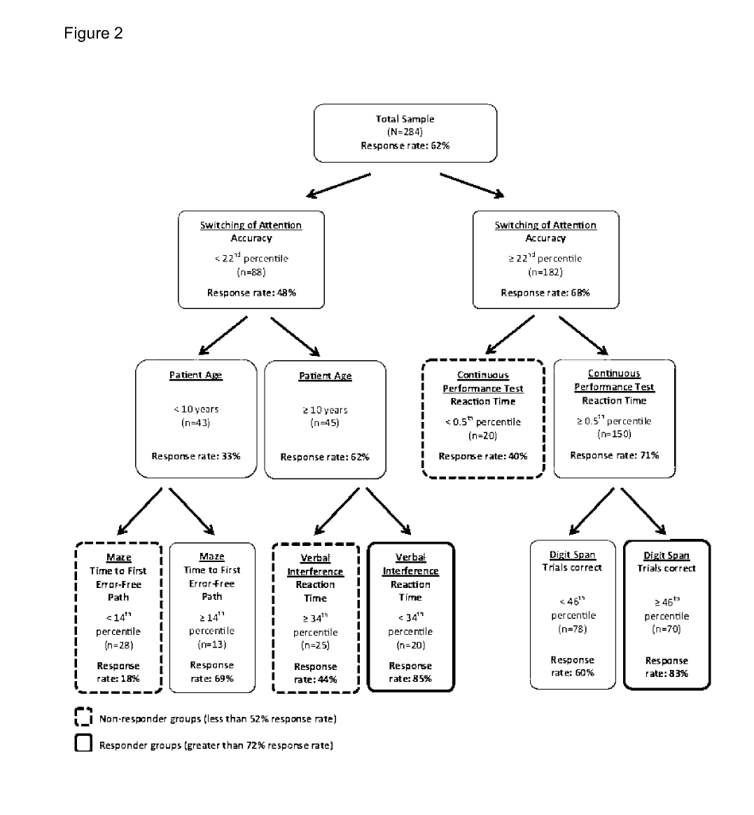

Of the 10 tasks (48 scores) in the cognitive test battery and 2 demographic measures of gender and age, 5 cognitive tasks were identified in the ROC tree of FIG. 2 as significant discriminators of responders and non-responders:

1.) the Switching of Attention accuracy,

2.) the reaction time on the Continuous Performance Test (n-back),

3.) the time to complete first Maze run without error,

4.) the Verbal Interference reaction time, and

5.) the Digit Span number of correct trials.

As shown in FIG. 2, a total of seven subgroups of patients were identified in the ROC analysis varying in their response to MPH treatment.

Among three groups of patients shown as gray boxes with dashed outlines in FIG. 2 (n=28; n=25; and n=20; grouped based on assessment using the above-described cognitive test battery) the proportions of responders to MPH were significantly lower (18%, 44%, and 40%) relative to the overall response rate of 62% (see FIG. 2).

Among two groups of patients shown as gray boxes with bolded outlines (n=20 and n=70; grouped based on assessment using the above-described cognitive test battery) the proportions of responders to MPH were significantly higher (85% and 83%) than the overall response rate (see FIG. 2).

Two groups of patients (n=13 and n=78; shown as white boxes in the lowest panel of FIG. 2) were identified that had responder rates comparable to the overall sample (69% and 60%).

Patients with Lower Response Rates to MPH

Based on assessment scores at baseline (patient responses to the 45-minute cognitive battery), ROC classifications identified three groups of children and adolescents with specific pre-treatment cognitive assessment profiles (n=28, n=25 and n=20), who have a significantly reduced response rate to MPH compared to the overall ADHD group response rate (“significantly” here refers to being “beyond the clinically significant cut-off point of 10 percentage points below the overall group response rate level of 62%”.

One of these groups consisted of patients with poor accuracy in Switching of Attention (their normative scores in the Switching of Attention accuracy test were below the 22nd percentile), who were younger than 10 years old and also had poor planning implementation (i.e. Maze scores below the 14^{th} percentile for the time taken to complete the first error-free path). This group of younger patients contributed the lowest proportion of responders with a response rate of only 18%.

The second group of patients identified as less likely to respond to MPH also had poor Switching of Attention scores (i.e. below the 22^{nd} percentile) but were 10 years of age or older and also scored in the normal to high range on the verbal interference task. These patients had verbal interference reaction times above or equal to the 34^{th} percentile relative to the normative population (n=25) and had a response rate of only 44%.

A third group of patients was identified using the scores on the Continuous Performance Test (CPT), a measure of sustained attention. Of the patients with normal or high Switching of Attention accuracy (in or above the 22^{nd} percentile; see FIG. 2, right side), those with very low sustained attention reaction time scores (n=20; CPT scores<0.5^{th} percentile), only 40% qualified as responders to MPH.

These three distinct cognitive pre-treatment profiles which identified three groups of patients with a reduced response rate to MPH (compared to the overall ADHD group response rate) are shown in gray boxes with dashed outlines in FIG. 2.

Patients with Higher Response Rates to Methylphenidate (MPH)

ROC signal detection also identified two groups of children and adolescents with distinct cognitive pre-treatment profiles in which these profiles correlated with a significantly increased response to MPH compared to the overall ADHD group response rate.

Eighty-five percent (85%) of participants responded to MPH if they were 10 years of age or older, and showed low cognitive flexibility as measured by the Switching of Attention test accuracy scores (<22^{nd} percentile) and poor Verbal Interference reaction times (<34^{th} percentile; a measure of impulsivity) at baseline.

A second pre-treatment profile corresponding with a high response rate (83%): was identified. Specifically, among participants with normal to above-normal Switching of Attention accuracy scores (>=22^{nd} percentile), and not having extremely slow Continuous Performance Test reaction time scores (>=0.5th percentile) and normal to above-average scores on the Digit Span task (>=46^{th} percentile). Among these patients (n=70), being accurate, fast, and having good working memory was associated with high response to MPH.

The resulting ROC classification tree comprised five group of patients with pre-treatment profiles that were associated with either significantly increased treatment response rates (two groups, together comprising 32% of the sample: 98/284) or significantly decreased treatment response rates (three groups together comprising 24% of the sample: 73/284). Using the using the percentile cut-off points identified by each ROC analysis to identify these groups of patients, the specificity of these classifications was 77% and had a negative predictive value of 67%, reflecting good utility for accurately identifying non-responders. In addition, these classifications yielded a sensitivity value of 76% and positive predictive value of 83%, reflecting good utility for accurately identifying responders.

Table 4 compares the low response groups and high response groups in relation to other parameters measured in the present study. Groups were found to statistically differ in age, which is expected since age forms part of the classification tree. Groups did not significantly differ with respect to:
gender;
baseline levels of overall ADHD symptom severity (as measured by the clinician rated ADHD-RS-IV, or parent rated CPRS-R:L);
the dosage of MPH at the time of post-treatment assessment (week 6);
baseline levels of overall ADHD symptom severity (as measured by the clinician rated ADHD-RS-IV, or parent rated CPRS-R:L); or
proportions of the clinical combined or hyperactive ADHD sub-types, or the inattentive ADHD subtype.

Showing that these criteria are not indicators for the above-described clinical or demographic features defining the patient groups identified.

TABLE 4
Comparison of Low Responders, Average Responders and High
Responders on Clinical and Demographic Parameters

| | Responder group | | | Low | | Average vs. | |
|---|---|---|---|---|---|---|---|
| | Low | Average | High | vs. High | | High | |
| | Responders | Responders | Responders | t or x² | p | t or x² | p |
| N | 74 | 91 | 90 | | | | |
| Mean Age in | 11.3 (3.1) | 11.8 (3.2) | 12.9 (2.9) | −3.22 | .002 | −2.23 | 0.02 |
| years (sd) | | | | | | | |
| % Male | 73.0 | 75.8 | 71.1 | 0.07 | 0.79 | 0.52 | 0.47 |
| Baseline Mean | 37.4 (11) | 35.6 (10) | 36.6 (10) | 0.53 | 0.60 | −0.63 | 0.53 |
| ADHD-RS-IV^{b} | | | | | | | |
| (sd) | | | | | | | |
| Baseline Mean | 72.4 (13) | 71.9 (13) | 74.7 (12) | −1.18 | 0.24 | −1.55 | 0.12 |
| Conners CGI^{c} | | | | | | | |
| (sd) | | | | | | | |
| Combined or | 64% | 54% | 56% | 1.06 | 0.30 | 0.53 | 0.82 |
| Hyperactive | | | | | | | |
| Subtype | | | | | | | |
| Inattentive | 30% | 37% | 39% | 1.50 | 0.22 | 0.45 | 0.83 |
| Subtype | | | | | | | |
| Mean Dose at 6 | 21.6 (16) | 22.8 (12) | 25.1 (17) | −1.36 | 0.18 | −1.04 | 0.30 |
| weeks (sd) | | | | | | | |
| Proportion of | 32% | 62% | 83% | | n/a | | n/a |
| Responders | | | | | | | |

^{a}Low responders = children and adolescents with response rates 10% or less than the base response rate (62%); High responders = children and adolescents with response rates 10% or higher than the base response rate (62%)
^{b}ADHD-RS-IV = ADHD Rating Scale IV;
^{c}Conners CGI = Conners Global Index (Conners Parent Rating Scale)

### DISCUSSION

In this large effectiveness study of the effects of methylphenidate on ADHD symptoms, the response to MPH was 62%, which is comparable with the literature (Kooij et al., 2010). Signal detection methods and ROC analyses were chosen for this planned hypothesis-generation that will guide subsequent, formal hypothesis testing.

Specifically, baseline responses to a computerized cognitive test battery comprising 10 tests yielding 48 scores were evaluated for their ability to differentiate responders and non-responders at 6 weeks of MPH treatment.

For the purpose of hypothesis-generation, baseline scores from an initial cohort were analysed using signal detection methods. Responses to the tasks of Switching of Attention, Continuous Performance Test, Verbal Interference, and Maze significantly predicted whether children and adolescents experienced a subsequent reduction of ADHD symptoms after receiving MPH, i.e. were useful biomarkers in the methods of the present invention. Specifically, the proportion of responders among those with the ROC-defined non-responder profiles was 33%, and the proportion of responders among the children with responder profiles was 83%. The effect size seen for utility of the test in ROC tree analyses shows that the algorithm derived from the cognitive tests is useful in clinical practice.

Non-responders had compromised cognition, including switching of attention, sustained attention, planning, and impulsivity. Responders showed intact cognition, particularly in speed and accuracy, switching of attention, sustained attention, and working memory. Response profiles yielded response rates ranging from 18% to 85%. Altogether, more than half of the sample could be placed into the much more or much less likely to respond groups defined a priori as entailing an effect size (Number Needed to Treat) of 10 or less.

Four of the most common findings in previous ADHD research in relation to ADHD symptom severity have included: a) deficits in sustained attention, measured herein using the Continuous Performance Test (Barkley 1997; Williams et al., 2010); b) deficits in working memory (via Digit Span) (Barkley 1997); c) increased impulsivity (reflected in Verbal Interference reaction times) (Lansbergen 2007; Williams et al., 2010); and d) frontal executive inefficiencies (Barkley 1997), exemplified by Maze errors and well as Switching of Attention inaccuracies.

Notwithstanding, the skilled reader will appreciate that neither of these cognitive parameters alone, or in combination, has previously been suggested or disclosed as being useful in patient-specific, cognitive pre-treatment profiles, i.e. as useful biomarkers, for establishing a cognitive pre-treatment profile allowing the prediction of MPH treatment outcome in children and adolescents suffering from ADHD, as is one embodiment of the present invention.

The Switching of Attention task, which was the first variable identified in the present study to discriminate MPH treatment responders and non-responders, requires holding “on-line” 2 sets of information at the same time and alternately switching between them, utilizing abilities of cognitive flexibility and executive function, both of which are deficits associated with ADHD. Greater errors on this task reflect a deficit in this ability, reflecting the continuation of using the same set of information, instead of switching to the alternate set of information.

The Continuous Performance (n-back) Task assesses the capacity to sustain attention over a prolonged period of time, and requires watching a series of letters appear on the screen, remembering each time what the previous letter was, in order to be able to respond when the same letter appears twice in a row. Poor performance in the Continuous Performance (n-back) Task is often seen in ADHD patients (Williams et al., 2010). Lower accuracy and slower response times reflect poorer attention skills. However, the use of this cognitive parameter in a multi-parameter cognitive pre-treatment profile, i.e. as a useful biomarker, for the prediction of MPH treatment outcome in children and adolescents suffering from ADHD, as described here for one embodiment of the present invention, has not previously been suggested or disclosed.

The Verbal Interference task assesses controlled attention similar to the Stroop test (Sacks, 1991). It requires the reading of a colour name word, but responding with the name of the font colour the word is written in. As will be understood, answering correctly requires the test subject to inhibit the automatic response of reading out word written. Lower accuracy and slower response times in this task indicate a patient's difficulty in inhibiting automatic responses. Inhibiting automatic behaviour is a core deficit in ADHD. Notwithstanding, the use of this cognitive parameter in a multi-parameter cognitive pre-treatment profile, i.e. as a useful biomarker, for the prediction of MPH treatment outcome in children and adolescents suffering from ADHD, as described here for one embodiment of the present invention, has not previously been suggested or disclosed.

Higher scores obtained in the Maze Test are indicative of the extended time periods required for successfully completing a maze and suggest a deficit in executive functioning and planning skills. Executive functioning and planning skills are cognitive functions that are not generally known to be particularly stimulant-responsive. Further, higher scores indicate a person's greater difficulty in choosing, trying, rejecting, and adapting to alternative courses of thought and action. In addition to the obvious social and academic consequences of such problems, the current study, for the first time, suggests that children and adolescents suffering from ADHD and obtaining higher Maze test scores are markedly less likely to respond adequately to MPH treatment—the current mainstay treatment for ADHD. While previous ADHD research demonstrated deficits in Executive Functions. The correlation between Maze Test scores and non-response to MPH treatment in children and adolescents suffering from ADHD, as described here for one embodiment of the present invention, has not previously been suggested or disclosed.

In the current study, signal detection methods and ROC analyses were chosen for transparent hypothesis-generation. For the optimal classification of non-responders and responders to MPH treatment in a group of children and adolescents suffering from ADHD, multiple ROC tests were conducted inflating type 1 error risk (i.e. evaluation of 48 measures at each level of the classification tree) The use of alpha=0.01 for each test provided some, albeit limited protection, from this concern. Furthermore, the present study includes the application of ROC analysis rather than standard analytical tools such as general linear models, or logistic regression. The inventor realised that, as ROC analysis is not mathematically compromised by issues such as multi-collinearity (i.e., highly correlated predictor variables) or the distributions of the residuals (Kiernan et al., 2001), and as it is ROC analysis can be applied when evaluating a large number of potential predictors of any binary outcome such as treatment response/no treatment response, it was particularly useful to analyse the 48 variables tested in each participant of the present study.

As will be appreciated, the surprising findings of the present study, for the first time, have allowed for the provision of low-cost, short and easily conducted tests to identify children and adolescents who are markedly less likely to have a positive treatment response to stimulant medication. The benefits of identifying children and adolescents with ADHD who will not respond adequately to stimulant medication will be manyfold but, predominantly, will serve to reduce the occurrence of unnecessary side effects of stimulant medication where therapeutic benefit is achieved while facilitating an earlier exploration of alternative treatments to benefit the patients and their families.

### REFERENCES

Polanczyk G, de Lima M S, Horta B L, Biederman J, Rohde L A: The worldwide prevalence of ADHD: a systematic review and metaregression analysis. Am J Psychiatry 2007, 164:942-948
Kooij S J, Bejerot S, Blackwell A, Caci H, Casas-Brugue M, Carpentier P J, Edvinsson D, Fayyad J, Foeken K, Fitzgerald M, Gaillac V, Ginsberg Y, Henry C, Krause J, Lensing M B, Manor I, Niederhofer H, Nunes-Filipe C, Ohlmeier M D, Oswald P, Pallanti S, Pehlivanidis A, Ramos-Quiroga J A, Rastam M, Ryffel-Rawak D, Stes S, Asherson P: European consensus statement on diagnosis and treatment of adult ADHD: The European Network Adult ADHD. BMC Psychiatry 2010, 10:67
Froehlich T E, Epstein J N, Nick T G, Melguizo Castro M S, Stein M A, Brinkman W B, Graham A J, Langberg J M, Kahn R S: Pharmacogenetic predictors of methylphenidate dose-response in attention-deficit/hyperactivity disorder. J Am Acad Child Adolesc Psychiatry 2011, 50:1129-1139.
Greenhill L L, Abikoff H B, Arnold L E, Cantwell D P, Conners C K, Elliott G, Hechtman L, Hinshaw S P, Hoza B, Jensen P S, March J S, Newcorn J, Pelham W E, Severe J B, Swanson J M, Vitiello B, Wells K: Medication treatment strategies in the MTA study: relevance to clinicians and researchers. J Am Acad Child Adolesc Psychiatry 1996, 35:1304-1313.
Lansbergen M M, Kenemans J L, van Engeland H (March 2007). “Stroop interference and attention-deficit/hyperactivity disorder: a review and meta-analysis”. Neuropsychology 21 (2): 251-62.
Williams L M, Rush A J, Koslow S H, et al. International Study to Predict Optimized Treatment for Depression (iSPOT-D), a randomized clinical trial: rationale and protocol. Trials 2011; 12: 4.
World Medical Association (2008). “World Medical Association Declaration of Helsinki ethical principles for medical research involving human subjects.” 59^{th} WMA General Assembly, Seoul, October 2008. Retrieved 22 Jul. 2013, from http://www.wma.net/en/30publications/10policies/b3/17c.pdf.
American Psychiatric Association. Diagnostic and statistical manual of mental disorders (4^{th} ed., text revision). Washington, D.C.: Author; 2000.
Conners C K. Conners' rating scales—Revised users manual. North Tonawanda, N.Y.: Multi-Health Systems Inc.; 1997.
Sheehan D V, Sheehan K H, Shytle R D, et al. Reliability and validity of the Mini International Neuropsychiatric Interview for Children and Adolescents (MINI-KID). J Clin Psychiatry 2010; 71(3): 313-326
Watters A J and Williams L M. Negative biases and risk for depression; integrating self-report and emotion task markers. Depress Anxiety 2011; 28(8): 703-718.
Rowe D L, Cooper N J, Liddell B J, Clark C R, Gordon E and Williams L M. Brain structure and function correlates of general and social cognition. J Integr Neurosci 2007; 6(1): 35-74.
Szabo' M. The short version of the Depression Anxiety Stress Scales (DASS-21): factor structure in a young adolescent sample. J Adolesc 2010; 33(1): 1-8.
McCrae R R and Costa P T. A contemplated revision of the NEO Five-Factor Inventory. Pers Indiv Differ 2004; 36(3): 587-596.
Varni J W, Seid M and Rode C A. The PedsQL: measurement model for the pediatric quality of life inventory. Med Care 1999; 37(2): 126-139.
Froehlich T E, Epstein J N, Nick T G, et al. Pharmacogenetic predictors of methylphenidate dose-response in attention-deficit/hyperactivity disorder. J Am Acad Child Adolesc Psychiatry 2011; 50(11): 1129-1139.e1122.
Williams L M, Hermens D F, Thein T, et al. Using brain-based cognitive measures to support clinical decisions in ADHD. Pediatr Neurol 2010; 42(2): 118-126.
Hermens D F, Williams L M, Clarke S, Kohn M, Cooper N and Gordon E. Responses to methylphenidate in adolescent AD/HD: evidence from concurrently recorded autonomic (EDA) and central (EEG and ERP) measures. Int J Psychophysiol 2005; 58(1): 21-33.
Williams L M, Hermens D F, Palmer D, et al. Misinterpreting emotional expressions in attention-deficit/hyperactivity disorder: evidence for a neural marker and stimulant effects. Biol Psychiatry 2008; 63(10): 917-926.
Korgaonkar M S, Cooper N J, Williams L M and Grieve S M. Mapping inter-regional connectivity of the entire cortex to characterize major depressive disorder: a whole-brain diffusion tensor imaging tractography study. Neuroreport 2012; 23(9): 566-571.
Korgaonkar M S, Grieve S M, Koslow S H, Gabrieli J D E, Gordon E and Williams L M. Loss of white matter integrity in major depressive disorder: evidence using tract-based spatial statistical analysis of diffusion tensor imaging. Hum Brain Mapp 2011; 32(12): 2161-2171.
Korgaonkar M S, Grieve S M, Etkin A, Koslow S H and Williams L M. Using standardized fMRI protocols to identify patterns of prefrontal circuit dysregulation that are common and specific to cognitive and emotional tasks in major depressive disorder: first wave results from the iSPOT-D study. Neuropsychopharmacology 2013; 38(5): 863-871.
Chang K, Nayar D, Howe M and Rana M. Atomoxetine as an adjunct therapy in the treatment of co-morbid attention-deficit/hyperactivity disorder in children and adolescents with bipolar I or II disorder. J Child Adolesc Psychopharmacol 2009; 19(5): 547-551.
Gordon E, Barnett K J, Cooper N J, Tran N and Williams L M. An “integrative neuroscience” platform: application to profiles of negativity and positivity bias. J Integr Neurosci 2008; 7(3): 345-366.
Gordon E, Cooper N, Rennie C, Hermens D and Williams L M. Integrative neuroscience: the role of a standardized database. Clin EEG Neurosci 2005; 36(2): 64-75.
Gill, D. M., Reddon, J. R., Stefanyk, W. O., & Hans, H. S. (1986). Finger tapping: Effects of trials and sessions. Perceptual & Motor Skills, 62(2), 675-678.
Adam, J. J., Paas, F. G., Buekers, M. J., Wuyts, I. J., Spijkers, W. A., & Wallmeyer, P. (1999). Gender differences in choice reaction time: evidence for differential strategies. Ergonomics, 42(2), 327-35.
Groth-Marnat, G., & Baker, S. (2003). Digit Span as a Measure of Everyday Attention: A Study of Ecological Validity. Perceptual & Motor Skills, 97(3), 1209-1218.
Logan, G. D., & Cowan, W. B. (1984). On the ability to inhibit thought and action: a theory of an act of control. Psychological Review, 91, 295-327.
Logan, G. D., Cowan, W. B., & Davis, K. A. (1984). On the ability to inhibit simple and choice reaction time responses: a model and a method. Journal of Experimental Psychology: Human Perception & Performance, 10, 276-291.
Logan, G. D. (1994). On the ability to inhibit thought and action: a users guide to the stop signal paradigm. In: Dagenbach, D., Carr, T. H. (Eds.), Inhibitory Processes in Attention, Memory and Language. Academic Press, San Diego, pp. 189-239.
Baddeley, A., Emslie, H., & Nimmo-Smith, I. (1993). The Spot-the-Word test: a robust estimate of verbal intelligence based on lexical decision. British Journal of Clinical Psychology, 32, 55-65.
Blumberg, S. J., Foster, E. B., Frasier, A. M., Satorius, J., Skalland, B. J., Nysse-Carris, K. L., . . . O'Connor, K. S. (2012). Design and operation of the National Survey of Children's Health, 2007. [Research Support, U.S. Gov't, P.H.S.]. Vital Health Stat 1(55), 1-149.
Strine T W, Lesesne C A, Okoro C A, McGuire L C, Chapman D P, Balluz L S, Mokdad A H. 2006. Emotional and behavioral difficulties and impairments in everyday functioning among children with a history of attention-deficit/hyperactivity disorder. Prey Chronic Dis., 3(2):A52.
Barkley, R & Cox, D. (2007). A review of driving risks and impairments associated with attention-deficit/hyperactivity disorder and the effects of stimulant medication on driving performance. Journal of Safety Research, 38(1), 113-128.
Sharma, A., & Couture, J. (2013). A Review of the Pathophysiology, Etiology, and Treatment of Attention-Deficit Hyperactivity Disorder (ADHD). Ann Pharmacother
Hermens, D. F., Rowe, D. L., Gordon, E., & Williams, L. M. (2006). Integrative neuroscience approach to predict ADHD stimulant response. Expert Rev Neurother, 6(5), 753-763.
Gill, D. M., Reddon, J. R., Stefanyk, W. O., & Hans, H. S. (1986). Finger tapping: Effects of trials and sessions. Perceptual & Motor Skills, 62(2), 675-678.
Adam, J. J., Paas, F. G., Buekers, M. J., Wuyts, I. J., Spijkers, W. A., & Wallmeyer, P. (1999). Gender differences in choice reaction time: evidence for differential strategies. Ergonomics, 42(2), 327-35.
Groth-Marnat, G., & Baker, S. (2003). Digit Span as a Measure of Everyday Attention: A Study of Ecological Validity. Perceptual & Motor Skills, 97(3), 1209-1218.
Crossen, J. R., & Wiens, A. N. (1994). Comparison of the Auditory Verbal Learning Test (AVLT) and California Verbal Learning Test (CVLT) in a sample of normal participants. Journal of Clinical and Experimental Neuropsychology, 16, 190-194.
Sacks, T. L., Clark, C. R., Pols, R., & Geffen, L. B. (1991). Comparability and stability of performance on six alternate forms of the Dodrill-Stroop Color-Word Test. The Clinical Neuropsychologist, 5, 220-225.
O'Donnell, J. P., MacGregor, L. A., Dabrowski, J. J., Oestreicher, J. M., & Romero, J. J. (1994). Construct validity of neuropsychological tests of conceptual and attentional abilities. Journal of Clinical Psychology, 50(4), 596-600.
Borgaro, S., Pogge, D. L., DeLuca, V. A., Bilginer, L., Stokes, J., & Harvey, P. D. (2003). Convergence of different versions of the Continuous Performance Test: Clinical and scientific implications. Journal of Clinical & Experimental Neuropsychology, 25(2), 283-292.
Logan, G. D., & Cowan, W. B. (1984). On the ability to inhibit thought and action: a theory of an act of control. Psychological Review, 91, 295-327.
Bowden, S. C. (1989). Maze learning: Reliability and equivalence of alternate pathways. Clinical Neuropsychologist, 3(2), 137-144.
Williams, Leanne M., Mathersul, Danielle, Palmer, Donna M., Gur, Ruben C., Gur, Raquel E. and Gordon, Evian (2009) Explicit identification and implicit recognition of facial emotions: I. Age effects in males and females across 10 decades, Journal of Clinical and Experimental Neuropsychology, 31:3, 257-277.
Mathersul, D., Palmer, D. M., Gur, R. C., Gur, R. E., Cooper, N., Gordon, E., & Williams, L. M. (2009). Explicit identification and implicit recognition of facial emotions: II. Core domains and relationships with general cognition. J Clin Exp Neuropsychol, 31(3), 278-291.
Williams, L. M., Mathersul, D., Palmer, D. M., Gur, R. C., Gur, R. E., & Gordon, E. (2009). Explicit identification and implicit recognition of facial emotions: I. Age effects in males and females across 10 decades. J Clin Exp Neuropsychol, 31(3), 257-277.
Greenhill, L. L., Findling, R. L., & Swanson, J. M. (2002). A Double-Blind, Placebo-Controlled Study of Modified-Release Methylphenidate in Children With Attention-Deficit/Hyperactivity Disorder. Pediatrics. 109(3).
Pliszka, S. (2007). Practice Parameter for the Assessment and Treatment of Children and Adolescents With Attention-Deficit/Hyperactivity Disorder. Journal of the American Academy of Child and Adolescent Psychiatry, 46(7), 894-921.
Pappas, D. (2006). ADHD Rating Scale-IV: Checklists, Norms, and Clinical Interpretation. Journal of Psychoeducational Assessment. 24(2), 172-178.
Kiernan, M., Kraemer, H. C., Winkleby, M. A., King, A. C., Taylor, C. B. (2001). Do logistic regression and signal detection identify different subgroups at risk? Implications for the design of tailored interventions. Psychological Methods, 6(1): 35-48.
Kraemer H C (1999). Evaluating Medical Tests, Sage Publications, Newbury Park, Calif.
Barkley R A. Behavioral inhibition, sustained attention, and executive functions: constructing a unifying theory of ADHD. Psychological Bulletin. 1997; 121:65-94.
Williams L M, Hermens D F, Thein T, Clark C R, Cooper N J, Clarke S D, Lamb C, Gordon E, Kohn M R. Using brain based cognitive measures to support clinical decisions in ADHD. Pediatric Neurology 2010; 42:118-126.

1. A method of predicting a treatment outcome in a patient with attention-deficit/hyperactivity disorder (ADHD) comprising the steps of:
a) using a computer to assess a cognitive parameter in said patient thereby obtaining an assessment score for said parameter; and
b) comparing said assessment score of step a) with a reference set of assessment scores for said parameter to establish a correlation between said assessment score with a corresponding assessment score of the reference set, wherein said corresponding assessment score is linked to a treatment outcome in ADHD patients having been treated with a selected stimulant medication,
wherein said correlation of step b) is used to predict a treatment outcome for said patient with ADHD when treated with the selected stimulant medication.

2. The method of claim 1 wherein the stimulant medication is methylphenidate (MPH).

3. The method of claim 1 wherein said patient is aged between 6 and 17 years.

4. The method of claim 1 wherein the cognitive parameter is assessed by applying a computerised test battery including tests selected from Motor Tapping, Choice Reaction Time, Memory recall, Digit Span, Verbal Interference, Switching of Attention, Continuous Performance Test, Go/No-Go, Maze task and Emotion Identification.

5. The method of claim 1 wherein a first cognitive parameter is a predictor of negative treatment outcome for ADHD patients in one of the patient sub-groups when the division into said sub-groups is based on the comparison of at least a second cognitive parameter.

6. The method of claim 5 wherein said first cognitive parameter is high performance in the Switching of Attention test and wherein said second cognitive parameter is poor performance in the Continuous Performance Test.

7. The method of claim 5 wherein said first cognitive parameter is low performance in the Switching of Attention test and wherein said second cognitive parameter is poor performance in the Maze test or the Verbal interference test.

8. The method of claim 1 wherein a first cognitive parameter is a predictor of positive treatment outcome for ADHD patients in one patient sub-groups when the division into sub-groups is based on the comparison of at least a second cognitive parameter.

9. The method of claim 8 wherein said first cognitive parameter is poor performance in the Switching of Attention test and wherein said second cognitive parameter is high performance in the Verbal Interference test.

10. The method of claim 8 wherein said first cognitive parameter is high performance in the Switching of Attention test and wherein said second cognitive parameter is high performance in the Digit Span test.

11. A method of predicting negative MPH treatment outcome in a patient with attention-deficit/hyperactivity disorder (ADHD) comprising the steps of:
a) using a computer to subject said patient to a battery of cognitive tests assessing cognitive parameters, wherein said battery includes at least the Switching of Attention test, the Maze test and the Verbal Interference test to obtain the patient's assessment scores for at least each of the Switching of Attention, the Maze test and the Verbal Interference test; and
b) comparing said assessment scores of step a) with a reference set of assessment scores for cognitive parameters including assessment scores for at least each of the Switching of Attention, the Maze test and the Verbal Interference test;
wherein,
(i) when it is established that the patient's assessment score for the Switching of Attention test is below a predetermined percentile of the assessment scores for the Switching of Attention test in the reference set, and
when the patient's assessment score for the Maze test is below a predetermined percentile of the assessment scores for the Maze test in the reference set or the patient's assessment score for the Verbal Interference test is below a predetermined percentile of the assessment scores for the Verbal Interference test in the reference set; or
(ii) when it is established that the patient's assessment score for the Switching of Attention test is above said predetermined percentile in the assessment scores for the Switching of Attention test in the reference set of (i) above, and
when the patient's assessment score for the Continuous Performance Test is below a predetermined percentile of the assessment scores for the Continuous Performance Test in the reference set,
a negative MPH treatment outcome for said patient with ADHD is predicted.

12. The method of claim 11 wherein said patient is aged between 6 and 17 years.

13. The method of claim 11 wherein the predetermined percentile of the assessment scores for the Switching of Attention test in item (i) is below, or in item (ii) is above, the 50^{th} percentile of the assessment scores for the Switching of Attention test in the reference set.

14. The method of claim 13 wherein the predetermined percentile of the assessment scores for the Maze test in item (i) is below the 50^{th} percentile of the assessment scores for the Maze test in the reference set.

15. The method of claim 13 wherein the predetermined percentile of the assessment scores for the Verbal Interference test in item (i) is above the 30^{th} percentile of the assessment scores for the Verbal Interference test in the reference set.

16. The method of claim 13 wherein the assessment scores for the Continuous Performance Test in item (ii) is below the 50^{th} percentile of the assessment scores for the Continuous Performance Test in the reference set.

17. A method of predicting positive MPH treatment outcome in a patient with attention-deficit/hyperactivity disorder (ADHD) comprising the steps of:
a) Using a computer to subject said patient to a battery of cognitive tests assessing cognitive parameters, wherein said battery includes at least the Switching of Attention test, the Verbal Interference test and the Digit Span test to obtain the patient's assessment scores for at least each of the Switching of Attention test, the Verbal Interference test and the Digit Span test; and
b) Comparing said assessment scores of step a) with a reference set of assessment scores for cognitive parameters including assessment scores for at least each of the Switching of Attention test, the Verbal Interference test and the Digit Span test;
wherein,
(i) when it is established that the patient's assessment score for the Switching of Attention test is below a predetermined percentile of the assessment scores for the Switching of Attention test in the reference set, and
when the patient's assessment score for the Verbal Interference test is below a predetermined percentile of the assessment scores for Verbal Interference test in the reference set; or
(ii) when it is established that the patient's assessment score for the Switching of Attention test is above said predetermined percentile in the assessment scores for the Switching of Attention test in the reference set of (i) above, and
when the patient's assessment score for the Digit Span test is above a predetermined percentile of the assessment scores for the Digit Span test in the reference set,
a positive MPH treatment outcome for said patient with ADHD is predicted.

18. The method of claim 17 wherein said patient is aged between 6 and 17 years.

19. The method of claim 17 wherein the predetermined percentile of the assessment scores for the Switching of Attention test in item (i) is below, or in item (ii) is above, the 50^{th} percentile of the assessment scores for the Switching of Attention test in the reference set.

20. The method of claim 19 wherein the predetermined percentile of the assessment scores for the Verbal Interference test in item (i) is below the 50^{th} percentile of the assessment scores for the Verbal Interference test in the reference set.

21. The method of claim 19 wherein the predetermined percentile of the assessment scores for the Digit Span test in item (ii) is above the 30^{th} percentile of the assessment scores for the Digit Span test in the reference set.

22. A method of treating attention-deficit/hyperactivity disorder (ADHD) in a patient, said method comprising the steps of:
a) using a computer to subject said patient to a battery of cognitive tests assessing cognitive parameters, wherein said battery includes at least the Switching of Attention test, the Verbal Interference test and the Digit Span test to obtain the patient's assessment scores for at least each of the Switching of Attention test, the Verbal Interference test and the Digit Span test;
b) comparing said assessment scores of step a) with a reference set of assessment scores for cognitive parameters including assessment scores for at least each of the Switching of Attention test, the Verbal Interference test and the Digit Span test; and
c) administering methylphenidate (MPH) when:
(i) it is established that the patient's assessment score for the Switching of Attention test is below a predetermined percentile of the assessment scores for the Switching of Attention test in the reference set, and
when the patient's assessment score for the Verbal Interference test is below a predetermined percentile of the assessment scores for Verbal Interference test in the reference set; or
(ii) it is established that the patient's assessment score for the Switching of Attention test is above said predetermined percentile in the assessment scores for the Switching of Attention test in the reference set of (i) above, and
when the patient's assessment score for the Digit Span test is above a predetermined percentile of the assessment scores for the Digit Span test in the reference set.

23. The method of claim 22 wherein said patient is aged between 6 and 17 years.

24. The method of claim 22 wherein the predetermined percentile of the assessment scores for the Switching of Attention test in item (i) is below, or in item (i) is above, the 50^{th} percentile of the assessment scores for the Switching of Attention test in the reference set.

25. The method of claim 24 wherein the predetermined percentile of the assessment scores for the Verbal Interference test in item (i) is below the 50^{th} percentile of the assessment scores for the Verbal Interference test in the reference set.

26. The method of claim 24 wherein the predetermined percentile of the assessment scores for the Digit Span test in item (ii) is above the 30^{th} percentile of the assessment scores for the Digit Span test in the reference set.

27. The method of claim 22 wherein the cognitive parameter is assessed by applying a computerised test battery including tests selected from Motor Tapping, Choice Reaction Time, Memory recall, Digit Span, Verbal Interference, Switching of Attention, Continuous Performance Test, Go/No-Go, Maze task and Emotion Identification.
